(19) United States
(12) Patent Application Publication
(10) Pub. No.: US 2026/0159799 A1
(43) Pub. Date: Jun. 11, 2026

(54) OFF-GAS AND CONDENSATION MANAGEMENT FOR BIOREACTORS

(71) Applicant: Caladan Bio Inc., Brooklyn, NY (US)

(72) Inventors: Michael Kobida, JR., New York, NY (US); Robert Jauquet, Jersey City, NJ (US)

(21) Appl. No.: 19/181,763

(22) Filed: Apr. 17, 2025

Related U.S. Application Data
(60) Provisional application No. 63/635,077, filed on Apr. 17, 2024.

(51) Int. Cl. C12M 1/34; C12M 1/00

(52) U.S. Cl. CPC C12M 41/34; C12M 23/38; C12M 29/04; C12M 41/12; C12M 41/26

(57) ABSTRACT

A bioreactor includes a housing defining a receiving space, a reactor tank with a removable lid configured to be removably placed within the receiving space, a liquid input line and a gas input line in fluid communication with the reactor tank, a first sensor coupled with the removable lid to measure sensor data associated with the internal volume, and an off-gas tube fluidly coupled with the internal volume through the removable lid and connected to a second sensor to measure sensor data associated with off-gas produced from the reactor tank. The housing includes a cooling trough to receive and cool the off-gas tube below ambient temperature. The first sensor includes a connector that mates with a corresponding connector on the housing when the reactor tank is inserted into the receiving space.

### CROSS-REFERENCE TO RELATED APPLICATIONS

This application claims priority from U.S. Provisional Application No. 63/635,077, filed Apr. 17, 2024, and titled “CONDENSING AND OFF GAS MEASUREMENT IN BIOREACTOR SYSTEMS”, the entire disclosure of which is incorporated by reference for all purposes.

### TECHNICAL FIELD

This application relates generally to bioreactor vessels and more particularly to monitoring and sensing conditions within a bioreactor vessel for off-gas sensing and detection.

### BACKGROUND

A bioreactor refers to a device that supports the cultivation and processing of biological materials, such as cells, microorganisms, and biologically derived products. Typically, a bioreactor is a vessel in which a chemical process is carried out which involves organisms or biochemically active substances derived from such organisms. Cell cultures, consisting of cells growing suspended in a growth media, or on the surface of suspended particles, in solution, are produced within bioreactors with careful control of several parameters. Within a bioreactor, it is important to carefully control the environment to which the cells are exposed. Subtle changes in the environment may have major effects on the physiology of the cells and the amount of the target product. This in turn has a major impact on the economics of the production process. This process may either be aerobic or anaerobic. These bioreactors are commonly cylindrical, ranging in size from liters to cubic meters.

Bioreactors are vital tools in various industries, including biotechnology, pharmaceuticals, and industrial microbiology. Bioreactors are essential for the controlled cultivation and production of a wide array of biological materials, such as therapeutic proteins, vaccines, antibiotics, and biofuels. Single-use bioreactors have gained popularity due to their advantages, such as reduced contamination risk and simplified maintenance; however, traditional single-use bioreactors face several limitations, particularly concerning real-time monitoring and measurement capabilities.

In order to better understand the reaction that is taking place in the bioreactor, the gas coming out of the process occurring within the bioreactor may be measured. Typical measurements include carbon dioxide (CO₂) and oxygen (O₂). In order to take these measurements, a CO₂/O₂ off-gas meter is connected to the bioreactor. One challenge in taking these measurements is that the off-gas meter may only measure gas with a relative humidity of 90% or less. Gas typically coming out of a bioreactor is close to 100% humidity due to the environment within the bioreactor.

Therefore, there is a need for cost effective bioreactors and systems to integrate off-gas sensing systems capable of measuring gases from a bioreactor system that has a high level of relative humidity.

### SUMMARY

Various implementations of the present disclosure relate to bioreactor systems including continuous data monitoring, cost-efficiency, motion precision, and enhanced efficiency. Bioreactors and systems in accordance with the principles of the present description provide for ease of use and setup. Bioreactors and systems in accordance with the principles of the present description measure the gas coming out of the process cost-effectively and efficiently.

In accordance with the principles of the present description and an embodiment, a bioreactor includes a dissolved oxygen (DO) sensor, a pH sensor, and a conductivity sensor. The dissolved oxygen (DO) sensor, pH sensor, and conductivity sensor have connecting cables that terminate with respective connectors. The dissolved oxygen (DO) sensor, pH sensor, conductivity sensor, and connecting cables are installed into a plate. Cooperating connectors are secured in bioreactor housing. When being installed into bioreactor housing, the plate containing the dissolved oxygen (DO) sensor, the pH sensor, and the conductivity sensor connectors are positioned directly above cooperating connectors secured in the bioreactor housing. This provides for ease of use and setup.

In an embodiment of a bioreactor in accordance with the principles of the present description, the bioreactor housing defines a cooling trough. The cooling trough is designed to receive a tubing carrying off-gas from the bioreactor tank. The bioreactor housing further includes a cooling unit in operative connection with the cooling trough. The cooling unit removes heat from the bioreactor housing defining the cooling trough to cool the off-gas tubing in the cooling trough. This rapidly cools the off-gas, causing some of the moisture in the off-gas to condense to reduce the relative humidity. This enables the off-gas meter to measure gas coming out of the process cost-effectively and efficiently.

In an embodimen, the techniques described herein relate to a bioreactor including: a housing defining a receiving space; a reactor tank having a removable lid and defining an internal volume and configured to be removably placed within the receiving space; a liquid input line in fluid communication with the reactor tank to provide a liquid medium to the internal volume; a gas input line in fluid communication with the reactor tank to provide a gas to the internal volume; a first sensor coupled with the removable lid of the reactor tank and configured to measure sensor data associated with the internal volume; and an off-gas tube fluidly coupled with the internal volume through the removable lid and coupled to a second sensor configured to measure a sensor data associated with off-gas produced from the reactor tank.

In an embodiment, the first sensor includes at least one of a dissolved oxygen sensor, a pH sensor, or a conductivity sensor. In some aspects, the first sensor includes a first connector, the first connector having a vertical component extending along a first direction parallel with a direction for inserting the reactor tank into the receiving space; and the housing includes a second connector, the second connector configured to receive the first connector and provide an electrical connection as the reactor tank is inserted into the receiving space. In some aspects, the bioreactor further includes a filter component positioned adjacent the second sensor and wherein the off-gas tube is sloped, with respect to gravity, from a second end adjacent the second sensor to a first end adjacent the reactor tank. In some aspects, the housing further includes a cooling trough that receives the off-gas tube, wherein the cooling trough is configured to cool the off-gas tube below an ambient temperature. In some aspects, the cooling trough includes at least one of a Peltier cell, a fan, or a heat sink. In some aspects, the second sensor includes an off-gas meter configured to measure carbon dioxide and oxygen concentrations.

This summary of the description introduces concepts in a simplified form that are further described below in the Detailed Description. This Summary is not intended to identify key features or essential features of the claimed subject matter, nor is it intended to be used as an aid in determining the scope of the claimed subject matter.

### BRIEF DESCRIPTION OF THE DRAWINGS

The following figures, which form a part of this disclosure, are illustrative of described technology and are not meant to limit the scope of the claims in any manner.

FIG. 1 illustrates an example of a bioreactor system equipped with an off-gas collection and management system, according to an embodiment of this disclosure.

FIG. 2 illustrates a top view of a bioreactor system showing an off-gas and condensation management system, according to an embodiment of this disclosure.

FIG. 3 illustrates a perspective view of a bioreactor vessel with connections for sensors and management of the environment of the bioreactor vessel, according to an embodiment of this disclosure.

FIG. 4 illustrates a perspective view of a bioreactor vessel with connections for sensors and management of the environment of the bioreactor vessel, according to an embodiment of this disclosure.

FIG. 5 illustrates a perspective view of a portion of a bioreactor system with a bioreactor vessel installed in a bioreactor housing with sensor and management connections therebetween, according to an embodiment of this disclosure.

FIG. 6 illustrates a perspective view of a portion of a bioreactor system with a bioreactor vessel installed in a bioreactor housing showing an off-gas system in a cooling trough of the bioreactor housing, according to an embodiment of this disclosure.

FIG. 7 illustrates a portion of a bioreactor housing including a system for cooling the cooling trough, according to an embodiment of this disclosure.

### DETAILED DESCRIPTION

The following description sets forth exemplary aspects of the present disclosure. It should be recognized, however, that such description is not intended as a limitation on the scope of the present disclosure. Rather, the description also encompasses combinations and modifications to those exemplary aspects described herein.

Various implementations of the present disclosure will be described in detail with reference to the drawings, wherein like reference numerals present like parts and assemblies throughout the several views. In the figures, the left-most digit(s) of a reference number identifies the figure in which the reference number first appears. The use of the same reference numbers in different figures indicates similar or identical items. Furthermore, the drawings may be considered as providing an approximate depiction of the relative sizes of the individual components within individual figures. However, the drawings are not to scale, and the relative sizes of the individual components, both within individual figures and between the different figures, may vary from what is depicted. In particular, some of the figures may depict components as a certain size or shape, while other figures may depict the same components on a larger scale or differently shaped for the sake of clarity. Additionally, any samples set forth in this specification are not intended to be limiting and merely set forth some of the many possible implementations.

The present disclosure relates to bioreactor systems and methods for improving off-gas measurement and condensation management. Bioreactor systems may be used for cultivating and processing biological materials, such as cells, microorganisms, and biologically derived products. These systems may be employed in various industries, including biotechnology, pharmaceuticals, and industrial microbiology.

In some cases, bioreactor systems may include components for measuring off-gas produced during bioprocessing. However, measuring off-gas may present challenges due to high humidity levels in the gas exiting the bioreactor. Off-gas meters used in bioreactor systems may have limitations in their ability to accurately measure gases with high relative humidity. For example, some off-gas meters may only be capable of measuring gas with a relative humidity of 90% or less.

To address this challenge, bioreactor systems in accordance with the present disclosure may incorporate features for managing off-gas humidity levels. These features may allow for more accurate and reliable off-gas measurements, potentially improving process monitoring and control. By addressing the humidity limitations of off-gas meters, the disclosed bioreactor systems may provide enhanced capabilities for gas analysis in bioprocessing applications.

The bioreactor systems described herein may offer advantages in terms of ease of use, setup, and operational efficiency. These systems may integrate various components to facilitate off-gas measurement while managing condensation, potentially leading to improved bioprocess monitoring and control.

A system as described herein may include a bioreactor system that includes a bioreactor vessel contained within a housing and connected through one or more tubes to a source of nutrients for providing to the interior of the bioreactor vessel. Bioreactors include a reactor tank which typically is a cylindrical container. A liquid medium may be provided by a feeding pump via a liquid input line. Liquid input lines enable precise control over the addition of nutrients, supplements or other liquids required for the bioprocess. The control of fluids is important for maintaining optimal conditions. Gas may be provided by a gas pump via a gas input line. Gas input lines allow for the controlled delivery of gases, including oxygen and carbon dioxide, which are important for cellular respiration and pH regulation in the bioreactor.

In accordance with an aspect of the principles of the present invention, a single use bioreactor tank is designed to be delivered sterilized and ready to use with minimal setup. Bioreactors generally include a reactor tank which typically is a cylindrical container. A liquid medium may be provided by a feeding pump via a liquid input line. Liquid input lines enable precise control over the addition of nutrients, supplements or other liquids required for the bioprocess. This control is important for maintaining optimal conditions. Gas may be provided by a gas pump via a gas input line. Gas input lines allow for the controlled delivery of gases, including oxygen and carbon dioxide, which are important for cellular respiration and pH regulation in the bioreactor.

An agitator may be provided consisting of a motor driving a shaft having a plurality of mixing propellers positioned in the reactor tank. The inclusion of an agitator ensures thorough mixing of the biological media, promoting even distribution of nutrients and gasses. The agitator enhances the growth and productivity of the cultured organisms. Sensors connected to a system monitor, such as for example a pH sensor, a dissolved oxygen sensor, and a conductivity sensor may be provided. The pH sensor provides real-time monitoring and feedback on the acidity or alkalinity of the biological media. This information is important for maintaining the desired pH level throughout the bioprocessing. The dissolved oxygen sensor measures the concentration of oxygen dissolved in the biological media, providing important data for ensuring adequate oxygen supply to the cultured organisms. The conductivity sensor measures the ionic content of the biological media. The ionic variations of the medium over time may lead to changes in conductivity which would affect the measured impedance.

The bioreactor and system in accordance with the principles of the present invention integrate one or more sensors into the bioreactor unit for ease of use and setup. The bioreactor may include a main reactor tank body and a tank lid. Liquid input lines provide a liquid medium via a feeding pump into an interior of the bioreactor tank. The one or more sensors may include a temperature sensor, a dissolved oxygen (DO) sensor, a pH sensor, a conductivity sensor, and other such sensors that may be used to monitor a biological reaction and may be connected to a system monitor as described herein. A gas filter may be provided for sparging and overlay lines.

Connecting cables for the sensors including the temperature, dissolved oxygen (DO) sensor, pH sensor, and conductivity sensor are installed into a plate and conclude with respective connectors, such as male audio connectors. The bioreactor, plate, and connectors installs into the bioreactor housing in one movement for ease of use and setup as described below.

The bioreactor tank is installed by descending the bioreactor tank vertically into a tank housing defined in the bioreactor housing. The plate or lid containing the temperature sensors, dissolved oxygen (DO) sensor, the pH sensor, and conductivity sensor is positioned such that the connectors are positioned directly above corresponding connectors secured in the bioreactor housing. As the bioreactor tank descends vertically into tank housing, the sensor connectors are installed into the corresponding connectors in one movement to establish a connection between the sensors and the system monitor.

For measuring and detecting gases such as CO2/O2 an off-gas meter is connected to the bioreactor. When measuring the off-gas data, the meter may only be capable of measuring gas with a relative humidity of 90% or less. Gas coming out of a bioreactor is often close to 100% humidity. In order to eliminate some of this relative humidity, the off-gas is cooled before entering the off-gas meter. This cooling causes some of the moisture to condense from the gas which reduces the relative humidity.

To perform the cooling, the bioreactor housing includes a conductive casing that defines a cooling trough. The tubing carrying CO2/O2 off-gas from the tank is routed into the cooling trough. The off-gas tubing is connected to lid of the bioreactor tank and runs through a condensing unit. A filter is provided at an end of the off-gas tubing to maintain sterility in the tube.

The cooling trough may be cooled with a cooling unit such as a Peltier cooling pad, heat sink, and fan. The Peltier cooling pad in combination with the heat sink and fan remove heat from the conductive casing to cool the off-gas tubing in the cooling trough to rapidly cool off-gas. This causes some of the moisture to condense from the off-gas which reduces the relative humidity before entering the off-gas meter. The cooling and condensation enables the off-gas meter to measure gas coming out of the bioreactor process. The cooling trough and the off-gas tubing increase in elevation with respect to a gravitational reference as the gas line extends from the bioreactor. The elevational change allows condensed liquid in off-gas tubing to be pulled back into the bioreactor by gravity, to help maintain a constant volume of the liquid in the bioreactor to avoid inadvertently concentrating the biological media in the tank.

Continuous data monitoring enables real-time data acquisition which empowers scientists and operators to make precise, instantaneous adjustments to bioprocess conditions, leading to better control and process optimization. Cost-efficiency is achieved with the innovative shared use of one spectrometer across multiple bioreactors which significantly reduces integration costs, making advanced monitoring accessible to a broader range of applications. Motion precision is achieved utilizing the robotic features, which ensures consistent, accurate probe positioning, resulting in dependable measurements of cell media parameters throughout experiments. Enhanced efficiency results from automated data collection and integration which streamlines experiment management and decision-making, promoting higher productivity and resource utilization.

Thus, utilizing bioreactors and systems in accordance with the principles of the present description provide for ease of use and setup. Utilizing bioreactors and systems in accordance with the principles of the present description measure the gas coming out of the process cost effectively and efficiently. Utilizing bioreactors and systems in accordance with the principles of the present description offers an array of advantages over current bioreactors, systems, and bioprocess control, including continuous data monitoring, cost-efficiency, motion precision, and enhanced efficiency.

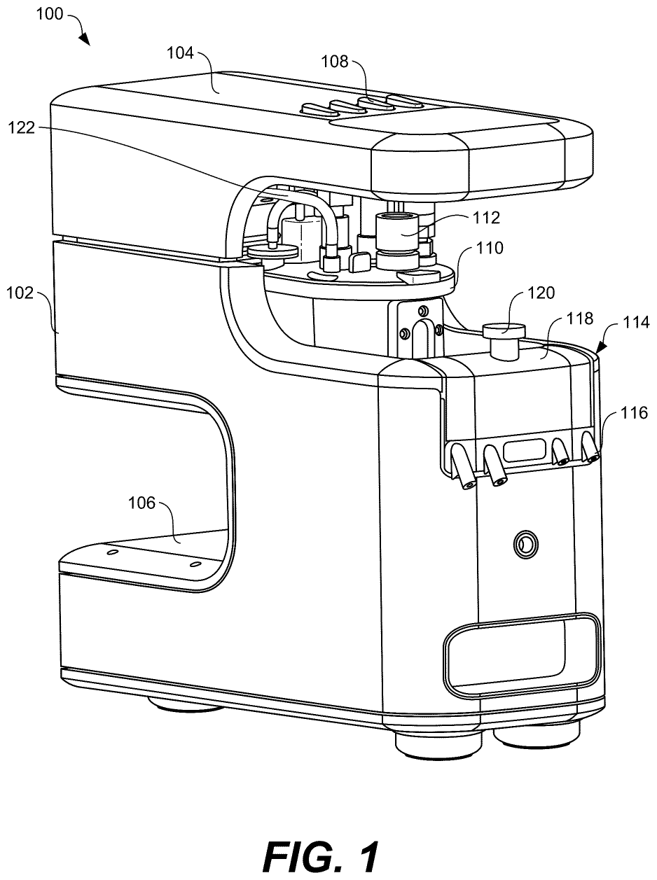

FIG. 1 illustrates an example of a bioreactor system 100 that includes a bioreactor tank having a sight glass for spectrometry and enabling optical sensing through the bioreactor system. FIG. 1 may illustrate an example of a bioreactor system 100 for use with optical spectroscopy, according to an example.

A bioreactor system 100 refers to a device that supports the cultivation and processing of biological materials, such as cells, microorganisms, and biologically derived products. Typically, a bioreactor includes a vessel 110 in which a chemical process is carried out which involves organisms or biochemically active substances derived from such organisms. This process may either be aerobic or anaerobic. These bioreactor vessels are commonly cylindrical, ranging in size from liters to cubic meters.

Bioreactor systems 100 are vital tools in various industries, including biotechnology, pharmaceuticals, and industrial microbiology. Bioreactor systems 100 are essential for the controlled cultivation and production of a wide array of biological materials, such as therapeutic proteins, vaccines, antibiotics, and biofuels. Single-use bioreactor systems have gained popularity due to their advantages, such as reduced contamination risk and simplified maintenance; however, traditional single-use bioreactors face several limitations, particularly concerning real-time monitoring and measurement capabilities.

The bioreactor system 100 includes a housing 102 that houses components of the system as well as a lid 104 that, when combined with the housing 102, encloses the vessel 110. The housing 102 may include and/or contain a controller, such as a computing device that may provide control of operations and devices as described herein. The housing 102 defines an imaging port 106 through which one or more optical sensors may detect characteristics or data relating to the contents of the bioreactor system 100. The imaging port 106 is shown as forming a passage into the housing 102. An additional port (not shown in FIG. 1) provides optical line of sight between a probe (e.g., Raman probe) and the sight glass of the reactor tank. One or more interfaces 108 on the lid 104 may be used to control operation of the bioreactor system 100, such as to control temperature, sample collection, nutrient delivery, timing, and other such parameters of the system contained within the bioreactor system 100.

The housing 102 further includes one or more components for receiving and collecting sensor data with respect to off-gas materials received through an off-gas system fluidly coupled with the vessel 110. The housing 102 may include an off-gas meter configured to measure carbon dioxide and oxygen in the off-gas from the vessel 110.

The vessel 110 includes a reactor tank which typically is a cylindrical container having a lid 112 that encloses a volume for containing and controlling an environment within the vessel 110. A liquid medium may be provided by a pump system 114 via one or more liquid input lines 116. Pumps included under cover 118 and secured in place by clip 120 may provide fluid into the vessel 110 through tubing. Liquid input lines 116 enable precise control over the addition of nutrients, supplements or other liquids required for the bioprocess within the vessel 110. This control is important for maintaining optimal conditions for a particular reaction or bioprocess. Gas may be provided by a gas pump via a gas input line. Gas input lines allow for the controlled delivery of gases, including oxygen and carbon dioxide, which are important for cellular respiration and pH regulation in the vessel 110.

The vessel 110 may be made from a material such as a metal or polycarbonate. And includes the lid 112 and a tank that together enclose a volume within the vessel 110. The interior of the vessel 110 may be measured using one or more sensors such as a dissolved oxygen sensor, pH sensor, conductivity sensor, and other such sensors. The sensors may be installed on the lid 112 of the vessel 110 and protrude into the interior of the vessel 110. The pH sensor provides real-time monitoring and feedback on the acidity or alkalinity of the biological media in the vessel 110. This information may be used for maintaining the desired pH level throughout the bioprocessing. The dissolved oxygen sensor measures the concentration of oxygen dissolved in the biological media, providing data for ensuring adequate oxygen supply to the cultured organisms. The conductivity sensor measures the ionic content of the biological media. The ionic variations of the medium over time may lead to changes in conductivity which would affect the measured impedance.

The one or more sensors are connected and/or coupleable (e.g., removable) from the vessel 110 for example through the lid 112 for ease of use and setup. The bioreactor unit may include a main reactor tank body and a lid 112. Liquid input lines provide a liquid medium via a feeding pump into an interior of the vessel 110. A gas filter may be provided for sparging and overlay lines.

Connecting cables for the sensors of the one or more sensors are installed into a plate and/or the lid 112 with connectors protruding in a vertical direction and oriented facing downward (with a gravitational reference frame). In an example, the connectors may include connectors that may slide into a corresponding connector fitting such as a male audio connector. In this manner, as the vessel 110 is installed in the housing 102 the connector installs, automatically, into a corresponding connector positioned on the housing 102 in one movement for ease of use and setup.

The vessel 110 is installed by descending the tank vertically into the housing 102 and specifically into a receive area defined in the housing 102. The lid 112 or another plat coupled to the vessel 110 that contains the temperature sensors, the dissolved oxygen (DO) sensor, the pH sensor, the conductivity sensor, and other such sensors is positioned such that the connectors are positioned directly above corresponding connectors secured in the housing 102. As the vessel 110 descends vertically into the housing 102 at the receiving area, the sensor connectors are installed into the corresponding connectors in one movement to establish a connection between the sensors and the system monitor.

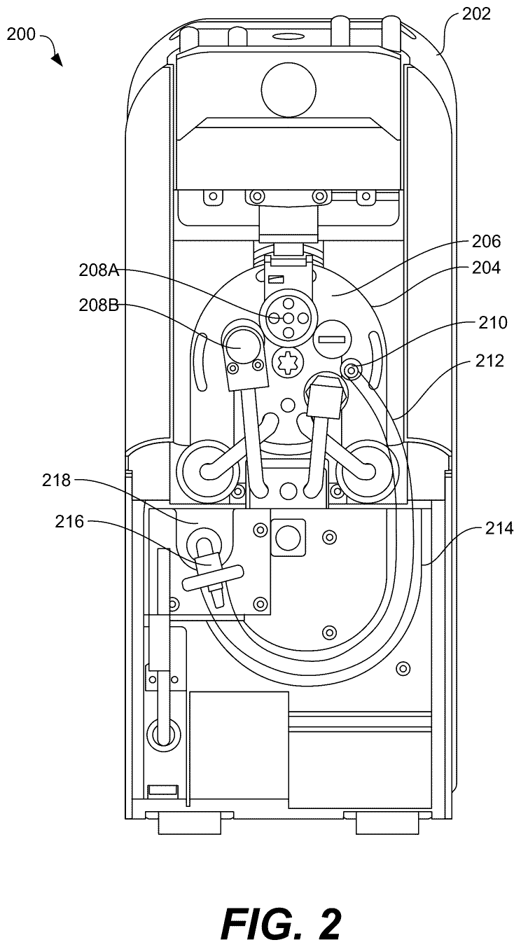

For measuring and detecting gases such as CO2/O2 an off-gas meter is connected to the bioreactor and stored within the housing 102. When measuring the off-gas data, the meter may only be capable of measuring gas with a relative humidity of 90% or less. Gas coming out of a bioreactor is often close to 100% humidity. In order to eliminate some of this relative humidity, the off-gas is cooled within the housing before entering the off-gas meter. This cooling causes some of the moisture to condense from the gas which reduces the relative humidity. The cooling portion of the housing 102 is shown and described in further detail with respect to FIGS. 2, 5, and 6 herein. To perform the cooling, the housing 102 includes a conductive casing that defines a cooling trough within the housing 102 to receive an off-gas tube 122. The tubing carrying CO2/O2 off-gas from the vessel 110 is routed into the cooling trough. The off-gas tubing is connected to lid 112 of the vessel 110 and runs through a condensing unit. A filter is provided at an end of the off-gas tubing to maintain sterility in the tube 122.

The cooling trough may be cooled with a cooling unit such as a Peltier cooling pad, heat sink, fan, and/or other such components. In an example, the Peltier cooling pad in combination with the heat sink and fan remove heat from the conductive casing to cool the off-gas tubing in the cooling trough to rapidly cool the off-gas. This causes some of the moisture to condense from the off-gas which reduces the relative humidity before entering the off-gas meter. The cooling and condensation enables the off-gas meter to measure gas coming out of the bioreactor process.

The cooling trough and the off-gas tubing increase in elevation with respect to a gravitational reference as the gas line extends from the vessel 110. The elevational change allows condensed liquid in off-gas tubing to be pulled back into the vessel 110 by gravity, to help maintain a constant volume of the liquid in the bioreactor to avoid inadvertently concentrating the biological media in the vessel 110.

FIG. 2 illustrates a top view of a bioreactor system 200 showing an off-gas and condensation management system, according to an embodiment of this disclosure. In some cases, a bioreactor system 200 may include a housing 202 that contains various components of the system. The housing 202 may be configured to enclose and support a reactor tank 204. A lid 206 may be positioned at the top of the reactor tank 204 to seal the contents within. The bioreactor system 200 may incorporate multiple sensors, such as a sensor 208A and a sensor 208B, which may be integrated into the system design. In some implementations, the sensors may include a temperature sensor, a dissolved oxygen sensor, a pH sensor, and a conductivity sensor. These sensors may be installed on the lid 206 of the reactor tank 204 and may protrude into the interior of the reactor tank 204.

An off-gas port 210 may be included in the bioreactor system 200, connecting to an off-gas tube 212 that extends through the system. The housing 202 may include a cooling trough 214 that accommodates the off-gas tube 212. In some cases, the off-gas tube 212 may connect to a filter 216 and ultimately lead to an off-gas meter 218. This arrangement of components may allow for the monitoring and measurement of gases produced during bioreactor operation. The bioreactor system 200 may also include a gas filter for sparging and overlay lines. These gas filters may help maintain the sterility of gases introduced into the reactor tank 204.

The configuration of the bioreactor system 200 may show how the off-gas tube 212 is routed through the cooling trough 214, potentially enabling temperature control of the gas as it flows from the reactor tank 204 to the off-gas meter 218. The filter 216 may be positioned along the off-gas tube 212 to help maintain system sterility during operation.

In some implementations, the off-gas tube 212 may be configured with a slope or incline from the filter 216 towards the reactor tank 204. This sloped configuration may serve multiple purposes within the bioreactor system 200. The incline may allow any condensed liquid that forms within the off-gas tube 212 to flow back into the reactor tank 204 due to gravitational forces. This feature may help maintain the liquid volume within the reactor tank 204, which may be important for preserving the concentration of the biological media and maintaining optimal conditions for the bioprocess.

The sloped arrangement of the off-gas tube 212 may also facilitate the movement of gases from the reactor tank 204 towards the off-gas meter 218. As the gases travel along the inclined path, they may encounter less resistance, potentially improving the efficiency of gas transfer and measurement. Additionally, the slope may help prevent the accumulation of liquid in any low points of the tubing, which could otherwise interfere with gas flow or measurements.

In some aspects, the degree of slope in the off-gas tube 212 may be adjustable or customizable based on the specific requirements of the bioprocess or the characteristics of the gases being produced. The housing 202 may include features or mechanisms that allow for the adjustment of the off-gas tube 212 angle, providing flexibility in system configuration and optimization. The combination of the sloped off-gas tube 212 and the cooling trough 214 may work synergistically to manage both gas flow and condensation. As the gases cool while traveling through the cooling trough 214, any resulting condensation may be directed back towards the reactor tank 204 by the slope of the tube, helping to maintain system balance and measurement accuracy.

In some implementations, the bioreactor system 100 may include additional components such as the pump system 114 and liquid input lines 116 for providing liquid medium to the vessel 110. The housing 102 may also include a cover 118 that may be secured using a clip 120, as shown in FIG. 1. The arrangement of these components in the bioreactor system 200 may provide for an integrated configuration where various elements are positioned to enable fluid handling, gas management, and monitoring capabilities. This design may facilitate efficient operation while maintaining the sterility and functionality of the bioreactor system.

In some cases, the bioreactor system 200 may include components for collecting and measuring off-gas produced during the bioprocess. FIG. 2 illustrates an example configuration of an off-gas collection and measurement system within the bioreactor system 200. The bioreactor system 200 may include an off-gas port 210 connected to the reactor tank 204. The off-gas port 210 may allow gases produced during the bioprocess to exit the reactor tank 204. In some implementations, an off-gas tube 212 may be connected to the off-gas port 210 to transport the off-gas from the reactor tank 204 to other components of the system.

The housing 202 of the bioreactor system 200 may incorporate a cooling trough 214. The cooling trough 214 may be designed to accommodate a portion of the off-gas tube 212. In some cases, the cooling trough 214 may be thermally conductive and may be coupled to a cooling unit. The cooling unit may remove heat from the cooling trough 214, thereby cooling the off-gas as it passes through the portion of the off-gas tube 212 within the cooling trough 214.

In some implementations, a filter 216 may be positioned along the path of the off-gas tube 212. The filter 216 may serve to maintain sterility of the system by preventing contaminants from entering the reactor tank 204 through the off-gas tube 212. Additionally, the filter 216 may help remove particulates or other unwanted substances from the off-gas before it reaches measurement components.

The off-gas collection system may culminate in an off-gas meter 218. The off-gas meter 218 may be configured to measure various characteristics of the off-gas, such as composition, flow rate, or other parameters relevant to monitoring the bioprocess. In some cases, the off-gas meter 218 may be specifically designed to measure carbon dioxide and oxygen levels in the off-gas.

In some cases, the bioreactor system 200 may include components for managing off-gas temperature and reducing humidity. FIG. 2 illustrates an example configuration of a cooling system within the bioreactor system 200. The housing 202 of the bioreactor system 200 may incorporate a cooling trough 214. In some implementations, the housing 202 may include a conductive casing that defines the cooling trough 214. The conductive casing may be made of a thermally conductive material to facilitate efficient heat transfer. The cooling trough 214 may be designed to accommodate a portion of the off-gas tube 212. In some cases, the cooling trough 214 may be thermally coupled to a cooling unit. The cooling unit may remove heat from the cooling trough 214, thereby cooling the off-gas as the off-gas passes through the portion of the off-gas tube 212 within the cooling trough 214.

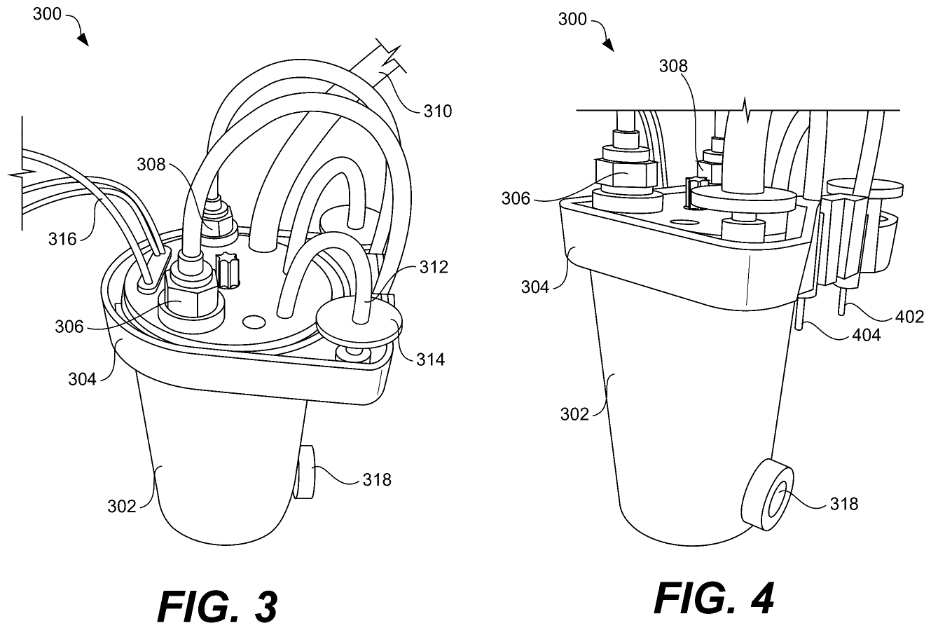

In FIG. 3 and FIG. 4, the reactor vessel 300 is shown as a component of the bioreactor system, such as the bioreactor system 200. In an embodiment, the reactor vessel 300 may be cylindrical in shape and is designed to contain the biological media and provide a controlled environment for bioprocessing. The reactor vessel 300 is installed within a bioreactor tank 302, which may serve as an outer shell or housing for the reactor tank.

The reactor vessel 300 is covered by a lid 304, which may be removable to allow access to the interior of the tank. The lid 304 may be equipped with various ports and connections to accommodate sensors and input/output lines. In the illustrated embodiment, multiple sensors (306, 308) are integrated into the lid 304, protruding into the interior of the reactor vessel 300. These sensors may include, but are not limited to, dissolved oxygen sensors, pH sensors, and temperature sensors.

The sensors may be removably inserted into the lid 304 in various ways to facilitate maintenance, replacement, and system flexibility. In some implementations, the lid 304 may include threaded ports that allow sensors to be screwed in and out as needed. These threaded connections may provide a secure and leak-proof seal while allowing for easy removal. In other aspects, the lid 304 may incorporate quick-connect fittings for rapid sensor installation and removal. These fittings may utilize a push-and-twist mechanism, allowing operators to insert or detach sensors without tools. The quick-connect design may include O-rings or gaskets to ensure a proper seal when the sensor is in place. The lid 304 may also feature keyed slots or bayonet-style mounts for sensor insertion. In this configuration, sensors may be equipped with corresponding tabs or protrusions that align with the keyed slots. Once inserted, the sensor may be rotated to lock it into position, providing a secure connection that may be easily disengaged when needed. In some cases, the lid 304 may employ a clamping mechanism for sensor attachment. This may involve a hinged or sliding clamp that secures the sensor in place when closed. The clamping mechanism may be designed to apply even pressure around the sensor, ensuring a tight seal and stable positioning. Another approach may involve using magnetic connections for sensor insertion. The lid 304 may contain embedded magnets or magnetic materials, with sensors designed to have complementary magnetic elements. This configuration may allow for easy alignment and attachment of sensors while providing a secure hold during operation. In some implementations, the lid 304 may incorporate a modular design with standardized sensor ports. These ports may accept interchangeable sensor modules that may be swapped out as needed. The modular approach may allow for greater flexibility in sensor selection and easier upgrades to new sensor technologies. The lid 304 may also feature a slide-in rail system for sensor insertion. In this design, sensors may be equipped with a compatible base that slides into guide rails integrated into the lid. The rails may guide the sensor into the correct position and provide a locking mechanism to secure it in place. In some aspects, the lid 304 may utilize a compression fitting system for sensor installation. This may involve a threaded collar that, when tightened, compresses a ferrule or O-ring around the sensor body, creating a secure and sealed connection. The compression fitting may allow for fine adjustments to sensor depth within the reactor vessel.

An off-gas tube 310 extends from the lid 304, allowing for the collection and routing of gases produced during the bioprocess. The reactor vessel 300 may also feature input lines (312, 316) that provide connections for introducing various substances such as nutrients, gases, or other additives into the tank. A filter 314 is incorporated into the system, which may help maintain sterility and control the introduction of gases or other materials into the reactor vessel 300. The reactor vessel 300 also includes a sight port 318, which allows for visual inspection and sensing of the contents without compromising the sealed environment.

In FIG. 4, additional details of the reactor vessel 300 are visible, including sensor connectors (402, 404) positioned on the lid 304. These connectors may facilitate the attachment and detachment of sensors or monitoring equipment, allowing for flexibility in system configuration and maintenance. The design of the reactor vessel 300, as shown in FIG. 3 and FIG. 4, may allow for efficient bioprocessing while providing multiple access points for monitoring, control, and maintenance of the bioreactor system. In some implementations, the sensor connectors on the reactor vessel may be designed to automatically mate with corresponding connectors in the housing as the reactor vessel is inserted. The sensor connectors may include male connectors that protrude vertically downward from the lid of the reactor vessel. These male connectors may be configured in various shapes such as pins, plugs, or bayonet-style fittings.

The housing may include female connectors positioned at the bottom of the receiving area, aligned to receive the male connectors from the reactor vessel. As the reactor vessel is lowered into the housing, the male connectors may slide into or engage with the female connectors, establishing electrical and/or data connections.

In some aspects, the connectors may incorporate a self-aligning feature, such as tapered edges or guide pins, to facilitate proper engagement even if there is slight misalignment during insertion. The connectors may also include a locking mechanism, such as a twist-lock or snap-fit design, which secures the connection once fully engaged. The housing connectors may be spring-loaded or mounted on flexible supports to accommodate slight variations in height or position, ensuring a secure connection is made. This flexibility may help prevent damage to the connectors during the insertion process. In some cases, the connectors may be designed with a keyed configuration to ensure that each sensor is connected to the correct corresponding port in the housing. This keying may prevent incorrect connections and simplify the setup process.

The connection system may also incorporate visual or audible indicators to confirm proper engagement. For example, the connectors may produce a clicking sound when fully seated, or may include LED indicators that light up when a successful connection is made. In some implementations, the connectors may be designed to be hot-swappable, allowing for sensor replacement or maintenance without powering down the entire system. This feature may enhance the flexibility and ease of use of the bioreactor system. The connector design may also incorporate sealing elements, such as O-rings or gaskets, to maintain the sterility of the bioreactor environment when the connections are made. These seals may help prevent contamination and ensure the integrity of the bioprocess.

In some implementations, the bioreactor system may incorporate an automatic gas connection mechanism that engages when the reactor vessel is inserted into the housing. This mechanism may include male gas connectors positioned on the bottom of the reactor vessel and corresponding female gas connectors located in the receiving area of the housing. The male gas connectors on the reactor vessel may be designed with a tapered or chamfered leading edge to facilitate smooth insertion. As the reactor vessel is lowered into the housing, these male connectors may align with and enter the female connectors. The female connectors may feature a funnel-like entry to guide the male connectors into place, accommodating slight misalignments during the insertion process.

The gas connectors may utilize a push-to-connect or quick-connect design that automatically seals the connection when fully engaged. This design may incorporate internal O-rings or gaskets that compress around the male connector as it is inserted, creating a gas-tight seal. The connectors may also include a locking mechanism, such as spring-loaded balls or clips, which engage once the connection is fully made, securing the gas line in place. In some aspects, the gas connection system may include multiple ports for different gas lines, such as sparging gas, overlay gas, or exhaust gas. These ports may be arranged in a specific pattern or use a keying system to ensure that each gas line connects to its intended counterpart, preventing incorrect connections. The housing may incorporate a slight vertical float or spring-loaded mounting for the female gas connectors. This feature may allow for small variations in height or alignment, ensuring that a proper connection is made even if there are minor manufacturing tolerances or wear over time.

To maintain system sterility, the gas connectors may include protective covers or seals that automatically retract or open during the connection process. These covers may help prevent contamination of the gas pathways when the reactor vessel is not installed in the housing. In some implementations, the gas connection system may include sensors or switches that detect when a proper connection has been made. These sensors may provide feedback to the control system, confirming that all gas lines are correctly connected before allowing the bioreactor process to begin. The automatic gas connection mechanism may be designed to withstand repeated connect/disconnect cycles without degradation of the sealing performance. Materials used in the connectors may be selected for their durability, chemical resistance, and compatibility with sterilization processes commonly used in bioreactor systems.

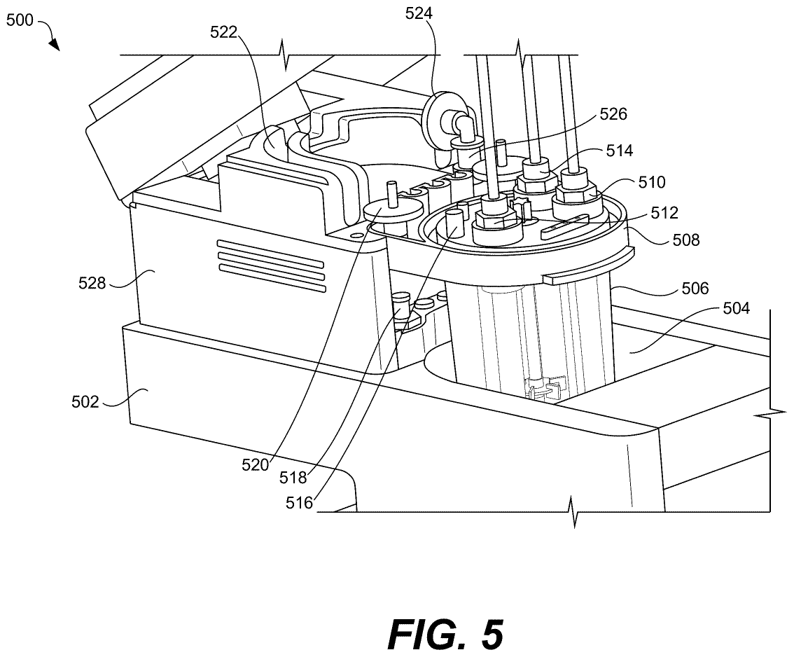

FIG. 5 provides another perspective of the off-gas collection and measurement system within a bioreactor assembly 500. The bioreactor assembly 500 may include similar components to those described in FIG. 2, arranged to facilitate efficient off-gas collection and measurement. In some cases, the bioreactor system 500 may include one or more sensors suite comprising multiple sensors for monitoring various parameters within the reactor tank 506. The one or more sensors may include a sensor 510, a sensor 512, and a sensor 514. These sensors may be positioned on the lid 508 of the reactor tank 506 and may protrude into the interior of the reactor tank 506 to measure different characteristics of the biological media.

The sensor 510 may be a dissolved oxygen (DO) sensor for measuring the concentration of oxygen dissolved in the biological media. The sensor 512 may be a pH sensor for monitoring the acidity or alkalinity of the media. The sensor 514 may be a conductivity sensor for measuring the ionic content of the biological media.

In some implementations, the sensors 510, 512, and 514 may have connecting cables that conclude with respective connectors. These connectors may be male audio connectors designed for easy installation and connection. The housing 502 of the bioreactor system 500 may include corresponding female audio connectors secured within the housing 502.

The bioreactor system 500 may be designed for vertical installation of the reactor tank 506 into the receiving area 504 of the housing 502. As the reactor tank 506 descends vertically into the receiving area 504, the male audio connectors of the sensors 510, 512, and 514 may align with and connect to the female audio connectors in the housing 502. This design may allow for automatic connection of the sensors to the system monitor in a single movement, facilitating ease of use and setup.

In some cases, the lid 508 or a plate attached to the reactor tank 506 may contain the sensors 510, 512, and 514 along with their connecting cables. The lid 508 or plate may be positioned such that when the reactor tank 506 is lowered into the receiving area 504, the connectors of the sensors are positioned directly above the corresponding connectors in the housing 502.

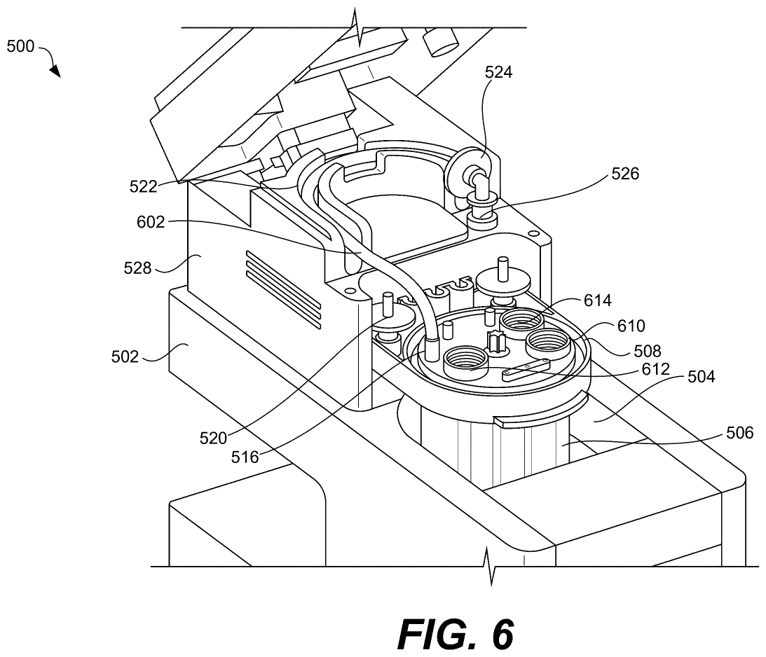

The bioreactor system 500 may also include sensor ports 610, 612, and 614 as shown in FIG. 6, arranged in the housing 502. These sensor ports may correspond to the sensors 510, 512, and 514 respectively, providing a connection point for the sensor connectors. A sensor connector 402 and a sensor connector 404 may be examples of the connectors used to establish the connection between the sensors and the system monitor. This configuration of the one or more sensors and connection mechanism in the bioreactor system 500 may allow for efficient installation and setup of the bioreactor, while ensuring proper connection of all sensors for accurate monitoring of the bioprocess parameters.

In the bioreactor assembly 500, an off-gas port 516 may be incorporated into the lid 508 of the reactor tank 506. The off-gas port 516 may connect to an off-gas tube that extends through the system. A filter 520 may be positioned along the off-gas flow path to maintain system sterility.

The bioreactor assembly 500 may include a cooling trough 522 integrated into the housing 502. The cooling trough 522 may be designed to receive a portion of the off-gas tube, allowing for temperature control of the off-gas as it flows through the system. A cooling unit 528 may be operatively connected to the cooling trough 522 to facilitate the cooling process.

FIG. 6 illustrates additional details of the off-gas collection and measurement system. In this view, an off-gas tube 602 is shown extending through the cooling trough 522. The arrangement of the off-gas tube 602 within the cooling trough 522 may allow for efficient cooling of the off-gas as it travels from the reactor tank 506 to the off-gas meter 526. In some implementations, the off-gas tube 602 may be configured with a slight incline as it extends from the reactor tank 506 towards the off-gas meter 526. This inclined configuration may serve multiple purposes. It may allow any condensed liquid that forms within the off-gas tube 602 to flow back into the reactor tank 506 due to gravitational forces, helping to maintain the liquid volume within the reactor tank 506. Additionally, the incline may facilitate the movement of gases from the reactor tank 506 towards the off-gas meter 526, potentially improving the efficiency of gas transfer and measurement. The combination of the cooling trough 522 and the inclined off-gas tube 602 may work together to manage both gas flow and condensation. As the gases cool while traveling through the cooling trough 522, any resulting condensation may be directed back towards the reactor tank 506 by the slope of the tube, helping to maintain system balance and measurement accuracy.

In some cases, a filter 524 may be positioned near the off-gas meter 526. This filter 524 may provide an additional level of filtration before the off-gas enters the measurement device, potentially improving the accuracy and reliability of the measurements.

The arrangement of these components in the bioreactor system may provide for an integrated configuration where various elements are positioned to enable efficient off-gas collection, cooling, and measurement. This design may facilitate effective monitoring of the bioprocess while maintaining system sterility and functionality.

In the bioreactor assembly 500, the cooling trough 522 may be integrated into the housing 502. The cooling trough 522 may be designed to receive a portion of the off-gas tube, allowing for temperature control of the off-gas as the off-gas flows through the system. A cooling unit 528 may be operatively connected to the cooling trough 522 to facilitate the cooling process. In some implementations, the cooling unit 528 may include a Peltier cooling pad. The Peltier cooling pad may be thermally coupled to the cooling trough 522 to provide active cooling. The cooling unit 528 may also include a heat sink and a fan. The heat sink may be thermally coupled to the Peltier cooling pad to dissipate heat, while the fan may provide forced air cooling to enhance heat dissipation from the heat sink.

FIG. 6 illustrates additional details of the cooling system. In this view, an off-gas tube 602 is shown extending through the cooling trough 522. The arrangement of the off-gas tube 602 within the cooling trough 522 may allow for efficient cooling of the off-gas as the off-gas travels from the reactor tank 506 to the off-gas meter 526. In some cases, the cooling trough 522 and the off-gas tube 602 may be configured with a slight incline as the cooling trough 522 and the off-gas tube 602 extend from the reactor tank 506 towards the off-gas meter 526. This inclined configuration may serve multiple purposes. The incline may allow any condensed liquid that forms within the off-gas tube 602 to flow back into the reactor tank 506 due to gravitational forces, helping to maintain the liquid volume within the reactor tank 506. Additionally, the incline may facilitate the movement of gases from the reactor tank 506 towards the off-gas meter 526, potentially improving the efficiency of gas transfer and measurement.

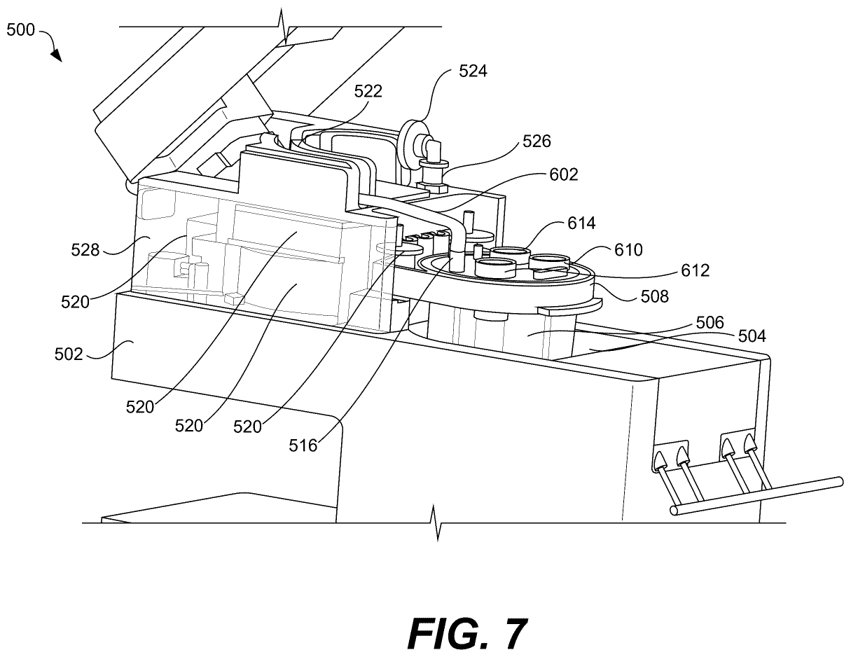

FIG. 7 provides a sectional side view of the bioreactor assembly 500, further illustrating the arrangement of the cooling trough 522 and the off-gas tube 602. The inclined configuration of the cooling trough 522 and the off-gas tube 602 is shown in this view. The combination of the cooling trough 522, the cooling unit 528, and the inclined off-gas tube 602 may work together to manage both gas temperature and condensation. As the gases cool while traveling through the cooling trough 522, any resulting condensation may be directed back towards the reactor tank 506 by the slope of the tube, helping to maintain system balance and potentially improving measurement accuracy.

Generally, for one or more of the embodiments described herein, numerous alternative arrangements may be described; it should be appreciated that these are presented for illustrative purposes only and are not limiting of the embodiments contained herein or the claims presented herein in any way. One or more of the arrangements may be widely applicable to numerous embodiments, as may be readily apparent from the disclosure. In general, arrangements are described in sufficient detail to enable those skilled in the art to practice one or more of the embodiments, and it should be appreciated that other arrangements may be utilized and that structural, logical, software, electrical and other changes may be made without departing from the scope or spirit of the present invention.

As used herein, the term “based on” may be used synonymously with “based, at least in part, on” and “based at least partly on.”

As used herein, the terms “comprises/comprising/comprised” and “includes/including/included,” and their equivalents, may be used interchangeably. An apparatus, system, or method that “comprises A, B, and C” includes A, B, and C, but also may include other components (e.g., D) as well. That is, the apparatus, system, or method is not limited to components A, B, and C.

Although the subject matter has been described in language specific to structural features and/or methodological acts, it is to be understood that the subject matter defined in the appended claims is not necessarily limited to the specific features or acts described.

### EXAMPLE CLAUSES

While the example clauses described above are described with respect to one particular implementation, it should be understood that, in the context of this document, the content of the example clauses may also be implemented via a method, device, system, computer-readable medium, and/or another implementation. Additionally, any of examples A-T may be implemented alone or in combination with any other one or more of the examples A-T.

A. A bioreactor that includes: a housing with a receiving space; a reactor tank with a removable lid that creates an internal volume and may be placed into and removed from the receiving space; a liquid input line connected to the reactor tank to supply liquid medium to the internal volume; a gas input line connected to the reactor tank to supply gas to the internal volume; a first sensor attached to the removable lid of the reactor tank that measures data from inside the internal volume; and an off-gas tube connected to the internal volume through the removable lid and connected to a second sensor that measures data from gases produced by the reactor tank.

B. The bioreactor of clause A, C, D, E, F, or G, where the first sensor is at least one of a dissolved oxygen sensor, a pH sensor, or a conductivity sensor.

C. The bioreactor of clause A, B, D, E, F, or G, where: the first sensor has a first connector with a vertical component that extends in the same direction as the reactor tank is inserted into the receiving space; and the housing has a second connector designed to receive the first connector and create an electrical connection when the reactor tank is placed into the receiving space.

D. The bioreactor of clause A, B, C, E, F, or G, with a filter component positioned next to the second sensor, and where the off-gas tube slopes downward from the second sensor end to the reactor tank end.

E. The bioreactor of clause A, B, C, D, F, or G, where the housing includes a cooling trough that holds the off-gas tube and cools it below the surrounding temperature.

F. The bioreactor of clause A, B, C, D, E, or G, where the cooling trough includes at least one of a Peltier cell, a fan, or a heat sink.

G. The bioreactor of clause A, B, C, D, E, or F, where the second sensor is an off-gas meter that measures carbon dioxide and oxygen concentrations.

H. A bioreactor system that includes: a reactor tank with a removable lid that creates an internal volume and may be placed into and removed from a receiving space in a housing; a first sensor attached to the removable lid of the reactor tank that measures data from inside the internal volume; and an off-gas tube connected to the internal volume through the removable lid and connected to a second sensor that measures data from gases produced by the reactor tank.

I. The bioreactor system of clause H, J, K, L, M, or N, where: the first sensor has a first connector with a vertical component that extends in the same direction as the reactor tank is inserted into the receiving space; and the housing has a second connector designed to receive the first connector and create an electrical connection when the reactor tank is placed into the receiving space.

J. The bioreactor system of clause H, I, K, L, M, or N, with a filter component positioned next to the second sensor, and where the off-gas tube slopes downward from the second sensor end to the reactor tank end.

K. The bioreactor system of clause H, I, J, L, M, or N, where the housing includes a cooling trough that holds the off-gas tube and cools it below the surrounding temperature.

L. The bioreactor system of clause H, I, J, K, M, or N, where the cooling trough includes at least one of a Peltier cell, a fan, or a heat sink.

M. The bioreactor system of clause H, I, J, K, L, or N, where the second sensor is an off-gas meter that measures carbon dioxide and oxygen concentrations.

N. The bioreactor system of clause H, I, J, K, L, or M, where the first sensor is at least one of a dissolved oxygen sensor, a pH sensor, or a conductivity sensor.

O. A bioreactor that includes: a housing with a receiving space; a reactor tank with a lid that creates an internal volume and may be placed into and removed from the receiving space; one or more input lines connected to the internal volume; a first sensor attached to the lid of the reactor tank that measures data from inside the internal volume; and an off-gas tube connected to the internal volume through the lid and connected to a second sensor at the far end of the off-gas tube that measures data from gases produced by the reactor tank.

P. The bioreactor of clause O, Q, R, S, or T, where the first sensor has a first connector with a vertical component that extends in the same direction as the reactor tank is inserted into the receiving space; and the housing has a second connector designed to receive the first connector and create an electrical connection when the reactor tank is placed into the receiving space.

Q. The bioreactor of clause O, P, R, S, or T, with a filter component positioned next to the second sensor, and where the off-gas tube slopes downward from the second sensor end to the reactor tank end.

R. The bioreactor of clause O, P, Q, S, or T, where the housing includes a cooling trough that holds the off-gas tube and cools it below the surrounding temperature.

S. The bioreactor of clause O, P, Q, R, or T, where the cooling trough includes at least one of a Peltier cell, a fan, or a heat sink.

T. The bioreactor of clause O, P, Q, R, or S, where the second sensor is an off-gas meter that measures carbon dioxide and oxygen concentrations.

What is claimed is:

1. A bioreactor comprising:
a housing defining a receiving space;
a reactor tank having a removable lid and defining an internal volume and configured to be removably placed within the receiving space;
a liquid input line in fluid communication with the reactor tank to provide a liquid medium to the internal volume;
a gas input line in fluid communication with the reactor tank to provide a gas to the internal volume;
a first sensor coupled with the removable lid of the reactor tank and configured to measure sensor data associated with the internal volume; and
an off-gas tube fluidly coupled with the internal volume through the removable lid and coupled to a second sensor configured to measure a sensor data associated with off-gas produced from the reactor tank.

2. The bioreactor of claim 1, wherein the first sensor comprises at least one of a dissolved oxygen sensor, a pH sensor, or a conductivity sensor.

3. The bioreactor of claim 1, wherein:
the first sensor comprises a first connector, the first connector having a vertical component extending along a first direction parallel with a direction for inserting the reactor tank into the receiving space; and
the housing comprises a second connector, the second connector configured to receive the first connector and provide an electrical connection as the reactor tank is inserted into the receiving space.

4. The bioreactor of claim 1, further comprising a filter component positioned adjacent the second sensor and wherein the off-gas tube is sloped, with respect to gravity, from a second end adjacent the second sensor to a first end adjacent the reactor tank.

5. The bioreactor of claim 4, wherein the housing further comprises a cooling trough that receives the off-gas tube, wherein the cooling trough is configured to cool the off-gas tube below an ambient temperature.

6. The bioreactor of claim 5, wherein the cooling trough comprises at least one of a Peltier cell, a fan, or a heat sink.

7. The bioreactor of claim 1, wherein the second sensor comprises an off-gas meter configured to measure carbon dioxide and oxygen concentrations.

8. A bioreactor system comprising:
a reactor tank having a removable lid and defining an internal volume and configured to be removably placed within a receiving space of a housing;
a first sensor coupled with the removable lid of the reactor tank and configured to measure sensor data associated with the internal volume; and
an off-gas tube fluidly coupled with the internal volume through the removable lid and coupled to a second sensor configured to measure a sensor data associated with off-gas produced from the reactor tank.

9. The bioreactor system of claim 8, wherein:
the first sensor comprises a first connector, the first connector having a vertical component extending along a first direction parallel with a direction for inserting the reactor tank into the receiving space; and
the housing comprises a second connector, the second connector configured to receive the first connector and provide an electrical connection as the reactor tank is inserted into the receiving space.

10. The bioreactor system of claim 8, further comprising a filter component positioned adjacent the second sensor and wherein the off-gas tube is sloped, with respect to gravity, from a second end adjacent the second sensor to a first end adjacent the reactor tank.

11. The bioreactor system of claim 8, wherein the housing further comprises a cooling trough that receives the off-gas tube, wherein the cooling trough is configured to cool the off-gas tube below an ambient temperature.

12. The bioreactor system of claim 11, wherein the cooling trough comprises at least one of a Peltier cell, a fan, or a heat sink.

13. The bioreactor system of claim 8, wherein the second sensor comprises an off-gas meter configured to measure carbon dioxide and oxygen concentrations.

14. The bioreactor system of claim 8, wherein the first sensor comprises at least one of a dissolved oxygen sensor, a pH sensor, or a conductivity sensor.

15. A bioreactor comprising:
a housing defining a receiving space;
a reactor tank having a lid and defining an internal volume and configured to be removably placed within the receiving space;
one or more input lines in fluid communication with the internal volume;
a first sensor coupled with the lid of the reactor tank and configured to measure sensor data associated with the internal volume; and
an off-gas tube fluidly coupled with the internal volume through the lid and coupled to a second sensor at a distal end of the off-gas tube configured to measure a sensor data associated with off-gas produced from the reactor tank.

16. The bioreactor of claim 15, the first sensor comprises a first connector, the first connector having a vertical component extending along a first direction parallel with a direction for inserting the reactor tank into the receiving space; and
the housing comprises a second connector, the second connector configured to receive the first connector and provide an electrical connection as the reactor tank is inserted into the receiving space.

17. The bioreactor of claim 15, further comprising a filter component positioned adjacent the second sensor and wherein the off-gas tube is sloped, with respect to gravity, from a second end adjacent the second sensor to a first end adjacent the reactor tank.

18. The bioreactor of claim 15, wherein the housing further comprises a cooling trough that receives the off-gas tube, wherein the cooling trough is configured to cool the off-gas tube below an ambient temperature.

19. The bioreactor of claim 18, wherein the cooling trough comprises at least one of a Peltier cell, a fan, or a heat sink.

20. The bioreactor of claim 15, wherein the second sensor comprises an off-gas meter configured to measure carbon dioxide and oxygen concentrations.
